(19) United States
(12) Patent Application Publication
(10) Pub. No.: US 2014/0379026 A1
(43) Pub. Date: Dec. 25, 2014

(54) DEVICES AND METHOD FOR TREATING WOUNDS

(71) Applicant: CURASEAL INC., Santa Clara, CA (US)

(72) Inventors: Harold F. CARRISON, Pleasanton, CA (US); Akshay MAVANI, Los Altos, CA (US); Stephen PIERALDI, San Bruno, CA (US)

(73) Assignee: CuraSeal Inc.

(21) Appl. No.: 14/309,242

(22) Filed: Jun. 19, 2014

Related U.S. Application Data
(60) Provisional application No. 61/837,598, filed on Jun. 20, 2013.

(51) Int. Cl. A61B 17/00

(52) U.S. Cl. CPC A61B 17/0057; A61B 2017/00646; A61B 2017/00637; A61B 2017/00641
USPC 606/213

(57) ABSTRACT

A healing device for sealing multiple fistulas, large-opening fistulas, and other wounds and for promoting tissue growth is coupled to tissue surrounding a first tissue opening. The healing device includes a first sealing member placed on a first surface of the tissue over the tissue opening. The healing device also includes a second sealing member placed over a second surface of the tissue over the tissue opening. The healing device may include a growth-enhancing layer located between the first sealing member and the second sealing member. The first sealing member and the second sealing member may sandwich the tissue in between to cover the tissue opening and may be held together with a plurality of securing members that do not exert any force on the tissue.

### CROSS-REFERENCE TO RELATED APPLICATION(S)

The present application claims priority to U.S. Provisional Patent Application No. 61/837,598, entitled “Devices and Methods for Treating Wounds,” filed on Jun. 20, 2013. The full disclosure of the above-listed patent application is hereby incorporated by reference herein.

### FIELD

The present disclosure relates generally to medical devices, and more specifically, to tissue opening or wound treatment devices.

### BACKGROUND

People may suffer from many different types of tissue damage. For example, tissue may be ripped, torn, abnormally formed, or otherwise punctured. Tissue damage may include a single opening in the tissue, which may vary in size, or may include multiple openings in the tissue. A larger tissue opening or wound may be harder to treat than a smaller tissue opening or wound—for example, it may be harder to encourage and protect tissue growth (e.g., from physical damage from outside or within the body, from biological assault, etc.). As an example, inserting sutures into tissue bordering the opening can further damage the tissue, preventing re-growth to fill the openings. Similarly, when a portion of tissue has multiple openings or wounds, each separate opening or wound may create treatment difficulties—for example, the openings or wounds may be spread out from one another and may be too small to treat on their own.

The information included in this Background section of the specification, including any references cited herein and any description or discussion thereof, is included for technical reference purposes only and is not to be regarded as subject matter by which the scope of the invention as defined in the claims is to be bound.

### BRIEF SUMMARY

One example of the disclosure may include a healing device for sealing or closing wounds or fistulas and promoting tissue growth. The healing device may be configured to be operably connected or coupled to tissue surrounding a tissue opening, such as a fistula. The healing device may include a first sealing member configured for placement over a first opening of at least one fistula in a first surface of the tissue. The first sealing member may be configured to be operably connected or coupled to the first surface of the tissue near the first opening and to be positioned at least partially over the first opening. The healing device may also include a second sealing member configured for placement over a second opening of the at least one fistula in a second opposing surface of the tissue. The second sealing member may be configured to be operably connected or coupled to the second surface of the tissue, such that an upper surface of the second sealing member is positioned at least partially over the tissue opening. The first and second sealing members may be configured to be operably connected or coupled together. The healing device may further include a growth-enhancing layer operably connected or coupled to, incorporated into, and/or blended into at least one of the first sealing member and the second sealing member (e.g., to at least a portion thereof). The growth-enhancing layer may be configured to be at least partially received within the tissue opening. In some embodiments, the growth-enhancing layer may comprise at least one polymer selected from the group consisting of collagen, polyglycolic acid (PGA), polylactic acid (PLA), poly-L-lactide acid (PLLA), polyglycolic-lactic acid (PGLA), poly-DL lactic acid (PDLLA), or a combination thereof.

In certain embodiments, a device may comprise more than one sealing member that is configured for placement over the at least one fistula in the first surface of tissue, and/or more than one sealing member that is configured for placement over the at least one fistula in a second opposing surface of the tissue. In some embodiments, a device may comprise more than one growth-enhancing layer.

Some embodiments of devices described herein may use one or more securing members, such as sutures, to help narrow a tissue opening, such as a fistula, or to hold internal material or a seal in place within a tissue opening. The securing members may, for example, be positioned within a fistula tract. Certain embodiments of devices described herein may comprise one or more tensioning members, such as one or more sutures (e.g., connected or coupled to one or more sealing members of a device). At least one of the tensioning members may be fixedly or slidably coupled to one or more sealing members of the device.

Certain embodiments of devices described herein may comprise a proximal sealing member and a distal sealing member, and one or more biomaterials coated or otherwise included on a proximal side of the distal sealing member and on a distal side and/or a proximal side of the proximal sealing member. Some embodiments of devices described herein may include at least one sealing member and at least one growth-enhancing layer operably connected or coupled to, incorporated into, and/or blended into a surface of the at least one sealing member.

Another example of the disclosure may include a closure device for sealing or closing a tissue wound. The device may include a first support surface configured to be operably connected or coupled to a first surface of a tissue near the wound. The first support surface may also be configured to at least partially cover a first region (e.g., opening) of the tissue wound. The device may also include a second support surface configured to be operably connected or coupled to the first support surface and the tissue near the wound. The second support surface may also be configured to at least partially cover a second region (e.g., opening) of the tissue wound. The device may further include a first layer configured to be operably connected or coupled to at least one of the first and second support surfaces. The first layer may comprise allograft and/or at least one polymer selected from the group consisting of collagen, polyglycolic acid, polylactic acid, poly-L-lactide acid, polyglycolic-lactic acid, poly-DL lactic acid, or a combination thereof.

In some embodiments, a wound closure device for sealing or closing a tissue wound may comprise a first sealing member configured for placement over a first region (e.g., opening) of the tissue wound and configured to extend beyond the tissue wound to cover margins of the tissue surrounding the tissue wound, a second sealing member configured for placement over a second region (e.g., opening) of the tissue wound and configured to extend beyond the tissue wound to cover margins of the tissue surrounding the tissue wound, and a first layer coupled to at least one of the first sealing member and the second sealing member, where the first layer comprises at least one polymer selected from the group consisting of collagen, polyglycolic acid (PGA), polylactic acid (PLA), poly-L-lactide acid (PLLA), polyglycolic-lactic acid (PGLA), poly-DL lactic acid (PDLLA), or a combination thereof.

In some embodiments, a wound sealing or closure device may comprise a sealing member, and a plurality of feet extending from a bottom side of the sealing member and comprising one or more bioabsorbable materials. The feet may be configured to be enveloped in and bond with tissue surrounding a wound after the wound sealing device is held over the wound and against the tissue under pressure for an extended period of time. The sealing member may comprise one or more bioabsorbable, biocompatible and/or implant-grade materials, and/or one or more resorbable materials that are capable of being resorbed into the tissue over time. In some cases, one or more bioabsorbable but non-implant-grade materials may be used. In some such cases, the non-implant-grade materials may, for example, be coated with one or more implant-grade materials.

In certain embodiments, a device for closing or sealing a wound or fistula may comprise a sealing member comprising an inner member and an outer member, with a suture configured to arrange the sealing member in a tissue opening, such as a fistula opening. The device may be sutured prior to, during, and/or after its installation.

A further example of the disclosure may include a method of closing or sealing a wound. The method may include sandwiching a tissue surrounding the wound between a first sealing member and a second sealing member. After the tissue is sandwiched between the two sealing members, tension may be provided to hold the tissue, the first sealing member, and the second sealing member together. Once under tension, the tissue, the first sealing member and the second sealing member may be operably connected or coupled together in a tensioned position.

In certain embodiments, a method of closing or sealing a wound may comprise positioning a section of tissue defining a wound between a first sealing member and a second sealing member, providing tension between the first sealing member and the second sealing member across the intermediate tissue, and coupling the first sealing member and the second sealing member together across the tissue to seal the wound and contact the intermediate tissue.

In some embodiments, a method of closing or sealing a wound may comprise positioning a section of tissue defining a wound between a first sealing member and a second sealing member, holding the first sealing member and the second sealing member in place across the intermediate tissue without applying tension to the intermediate tissue, and coupling the first sealing member and the second sealing member together across the tissue to seal the wound and contact the intermediate tissue.

Some embodiments of devices described herein may include sealing members comprising multiple layers and/or may comprise multiple layers of growth-enhancing materials. Certain embodiments of devices described herein may comprise a sealing member including a growth-enhancing material embedded or incorporated therein. In some embodiments, one or more tissue growth-promoting materials may be positioned between two sealing members. The tissue growth-promoting material(s) may be part of the device or may separate components that are later added to the device. In some cases, one or more devices and/or sealing members may be provided in a kit along with one or more tissue growth-promoting materials.

This summary is provided to introduce a selection of concepts in a simplified form that are further described below in the Detailed Description. This Summary is not intended to identify key features or essential features of the claimed subject matter, nor is it intended to be used to limit the scope of the claimed subject matter. A more extensive presentation of features, details, utilities, and advantages of the present invention as defined in the claims is provided in the following written description of various embodiments of the invention and illustrated in the accompanying drawings.

These and other aspects and embodiments will be described in further detail below, in reference to the attached drawing figures.

### BRIEF DESCRIPTION OF DRAWINGS

FIG. 1A is a front elevational view of a person having an abdominal tissue opening with a perforated intestine or bowel.

FIG. 1B is an enlarged isometric cross-sectional view of a single enteric perforation as shown in FIG. 1A.

FIG. 2 is an isometric, partially cross-sectional view of a healing device configured to cover at least a portion of a large tissue opening.

FIG. 3A is a cross-sectional view of the healing device of FIG. 2 operably connected to tissue margins surrounding a large tissue opening.

FIG. 3B is a top plan view of the healing device of FIG. 2 operably connected to tissue margins surrounding the large tissue opening.

FIG. 3C is a bottom plan view of the healing device of FIG. 2 operably connected to tissue margins surrounding the large tissue opening.

FIG. 4A is a top plan view of another exemplary embodiment of the healing device operably connected to tissue surrounding multiple tissue openings.

FIG. 4B is a cross-sectional view of the healing device of FIG. 4A operably connected to the tissue surrounding the tissue openings, viewed along line 4B-4B in FIG. 4A.

FIG. 5A is an enlarged isometric cross-sectional view of the healing device of FIG. 4A before being operably connected to the tissue, with an insertion shaft positioned within a plug aperture of the healing device.

FIG. 5B is an enlarged isometric cross-sectional view of the healing device of FIG. 4A before being operably connected to the tissue, with tensioning wires partially inserted within the insertion shaft.

FIG. 5C is an enlarged isometric cross-sectional view of the healing device of FIG. 4A operably connected to the tissue, with the tensioning wires in a full extended position.

FIG. 5D is an enlarged isometric cross-sectional view of the healing device of FIG. 4A operably connected to the tissue surrounding with a plug inserted within the plug aperture of the healing device.

FIG. 6A is an isometric view of another embodiment of the healing device.

FIG. 6B is a cross-sectional view of the healing device illustrated in FIG. 6A, viewed along line 6B-6B in FIG. 6A.

FIG. 7A is a top plan view of the healing device illustrated in FIG. 6A operably connected to tissue surrounding the tissue opening.

FIG. 7B is a cross-sectional view of the healing device of FIG. 6A operably connected to tissue surrounding the tissue opening viewed along line 7B-7B of FIG. 7A.

FIG. 8A is a top isometric view of another embodiment of a healing device.

FIG. 8B is a side elevational view of the healing device of FIG. 8A.

FIG. 8C is a bottom isometric view of the healing device of FIG. 8A.

FIG. 9 is a top isometric view of the healing device of FIG. 8A operably placed over a wound on a person.

### DETAILED DESCRIPTION

Some embodiments described herein comprise a device for covering and assisting in closing one or more tissue openings. Such a healing device may be used to cover tissue openings or tears and may be particularly effective for patching or closing relatively large wounds (e.g., wounds larger than 2 centimeters in diameter), as well as multiple wounds or small openings dispersed across a larger area. In one exemplary implementation, the healing device may include a first sealing member or first support surface and a second sealing member or second support surface. The first and second sealing members may be tensioned together via a tensioning member (e.g., a tensioning cord) operably connected to each sealing member. For example, the two sealing members may be positioned so that the first sealing member is located substantially over the second sealing member, with the damaged tissue at least partially positioned between the two sealing members. The tensioning member may then be pulled or otherwise tensioned, pulling the first and second sealing members together. Other components that may be pulled or otherwise tensioned may alternatively or additionally be used to bring the first and second sealing members toward each other.

The two sealing members may be secured in place in the tensioned position via securing members (e.g., sutures), which may be connected to the first and second sealing members through a portion of the tissue. The securing members holding the two sealing members together may be tensioned against the sealing members and not the tissue. In this way, the securing members may not pull or tug on the tissue, although a tensioning force is being exerted by the securing members through the tissue. This configuration may allow the healing device to cover the opening or wound without causing substantial damage to the tissue surrounding the opening. In other embodiments, the two sealing members may be secured together in correlative tension over a wound via a fastening or securing device. For example, the first sealing member may include a ball and stem, which may be inserted in a snap-fit configuration into a receiving cavity in the second sealing member. In some embodiments, sealing members may be held in place without the application of tension on the tissue therebetween. In certain embodiments in which tension is applied to sealing members, the tension may lessen over time (e.g., going to zero). This may happen, for example, if the tissue between the two members remodels and moves away from a location in which the tissue is under compression as a result of the tension.

In some embodiments, the tensioning wire may be used to press an interior sealing member against the tissue in order to hold the interior sealing member in place while it is connected to an exterior sealing member. In these embodiments, the tensioning wire (or wires) may comprise a resilient member having a hook shape on one end. The tensioning wire may be inserted into an opening within the tissue and an insertion aperture within each sealing member. The tensioning wire may be restrained in a generally straight configuration (e.g., through an introducer) as it is inserted through the tissue opening and the apertures in the sealing members. After the tensioning wire exits a bottom of the insertion aperture, the resilient member may return to a preformed hook shape to be positioned against a bottom surface of the second, interior sealing member. The tensioning wire may then be tensioned (e.g., by being pulled proximally by a surgeon), such that the hook is pressed against the bottom surface of the second, interior sealing member, thereby holding the interior sealing member in place against an interior wall of the tissue. The two sealing members may then be operably connected together (e.g., via sutures, stitches, etc.). The tensioning wires may hold the sealing members in a tensioned configuration around the tissue so that the sealing members can be connected together, but may be removed after the sealing members are secured in place.

The two sealing members may each include one or more layers of collagen and/or other tissue growth enhancers. The collagen and/or growth-enhancing layers may promote tissue growth on the two sealing members, thereby helping to fill or otherwise heal the opening. Additionally, the two sealing members may be positioned to cover or protect the opening to promote the healing and re-growth process. Further, in embodiments utilizing the tensioning wire, the collagen may be inserted as a plug into the insertion apertures, so that the openings in the tissue may be substantially filled.

In other embodiments, a single sealing member may be operably connected to tissue around a tissue opening. The sealing member may include bioabsorbable securing feet spaced intermittently around a bottom surface of the sealing member. The sealing member may then be positioned substantially over the opening, such that the securing feet may be positioned above non-damaged tissue (i.e., solid tissue). Once the sealing member is in position, the sealing member may be secured to the tissue (e.g., via tape, gauze, etc.). The feet may be configured to exert point pressure upon the skin or tissue of the user. As the feet press into the skin or tissue, the feet may be integrated with or enveloped into the skin. Thus, the sealing member may be substantially even with a first surface of the tissue as the feet “sink” into the tissue. In some embodiments, the sealing member and/or feet may be bioabsorbable so that they may eventually be absorbed into the skin or other tissue of the user as part of the healing process. The sealing member may further be coated with an environmentally resistant coating to aid in protecting the wound.

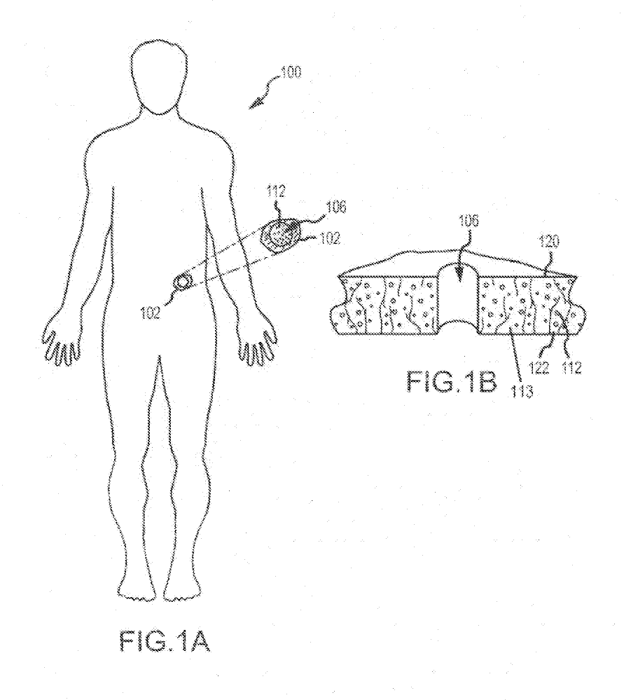

FIG. 1A is a front elevation view of a person 100 with an open wound 102 formed in his or her abdomen. The open wound 102 may be formed due to an injury, a genetic defect, surgery, surgical complications, and so on. While the open wound 102 is illustrated as being in the abdomen, the open wound 102 may be formed in nearly any part of the body of the person 100. In addition, the person 100 is depicted as having a perforated intestine or bowel, which is often referred to as a short-tract fistula 106. The fistula 106 is an enteroatmospheric fistula, as it is exposed to the atmosphere due to the abdominal wound 102. FIG. 1B is a cross-sectional view of the intestinal tissue 112 and the fistula 106. As can be seen in FIG. 1B, the fistula 106 may extend completely through the layer 113 of the intestinal tissue 112, or the fistula 106 may extend only partially through the layer 113. In this example, there is one fistula 106 in the enteric tissue 112 over a large, but cohesive, area. Alternatively, multiple fistulas or wounds in the intestine or other tissue 112 may present a need for closure.

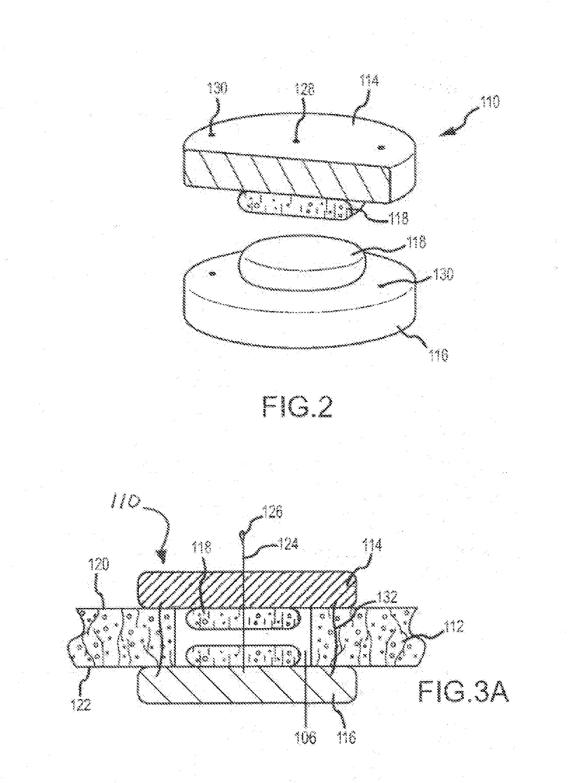

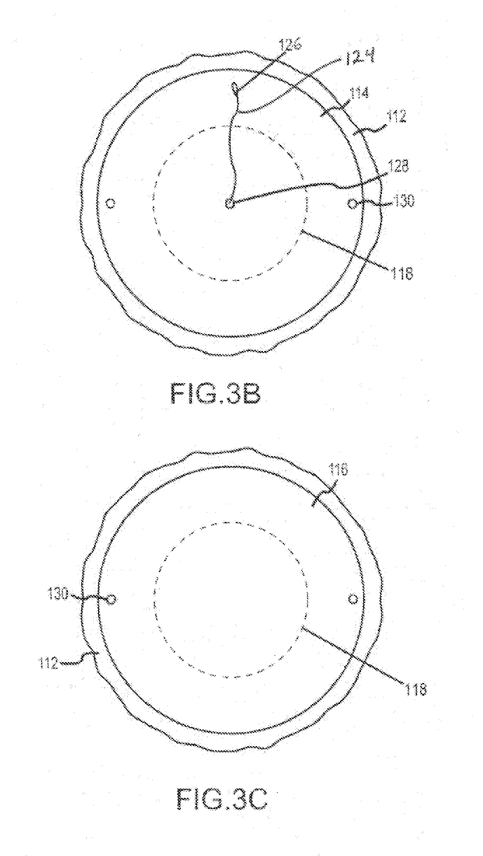

As shown in FIGS. 2 and 3A, a healing device 110 may be provided to promote healing by a large fistula opening 106 in tissue 112 and/or may provide protection for the fistula 106. For example, the healing device 110 may provide a growth framework 118 for re-growth of the tissue 112 therethrough, such that the fistula 106 may permanently close. FIG. 2 is an isometric view of the healing device 110. As shown there, the healing device 110 may include a first or exterior sealing member 114 that also functions as a first support surface and a second or interior sealing member 116 that also functions as a second support surface. The first and second sealing members 114, 116 may have substantially the same dimensions and/or may be customized depending on the size of the large-opening fistula 106. For example, the first and second sealing members 114, 116 may be configured to substantially cover openings of the fistula 106, as well as an area and a portion of the tissue 112 bordering or generally surrounding the fistula 106. As shown in FIG. 2, the sealing members 114, 116 may be generally shaped as cylindrical discs; however, other shapes, sizes and dimensions are possible. The shape and size of each sealing member 114, 116 may be determined based on the size, type, and location of the large-opening fistula 106. For example, the sealing members 114, 116 may be configured to generally trace the shape of the large-opening fistula 106, but each may have a larger diameter than the large-opening fistula 106.

With continued reference to FIG. 3A, in some embodiments, the first sealing member 114 may be positioned on a first (e.g., proximal) surface 120 of the tissue 112 and the second sealing member 116 may be positioned substantially parallel to the first sealing member 114 on a second (e.g., distal) surface 122 of the tissue 112. The second sealing member 116 (or interior member) may be compressed, folded, or rotated and then may be inserted into the large-opening fistula 106. The sealing members 114, 116 may include a resilient material that may be able to spring back to its original shape after deformation and insertion.

The first and second sealing members 114, 116 may comprise one or more bioabsorbable materials and/or may comprise a substantially fluid-impermeable coating on an outer surface. The bioabsorbable material or materials may allow the sealing members 114, 116 to eventually be absorbed by the body of the person 100, so that the sealing members 114, 116 may not have to be removed. The impermeability of an outer surface of the sealing members 114, 116 may allow the sealing members 114, 116 to act as a seal for the large-opening fistula 106, so that bacteria, fluids, and other potentially harmful substances may be substantially prevented from entering or exiting the fistula 106. In this manner, the sealing members 114, 116 may function as a protective cover for the fistula 106.

As shown in FIG. 2, the first sealing member 114 may include a tensioning cord aperture 128 for receiving a portion of a tensioning cord therethrough. Additionally, the first and second sealing members 114, 116 may include one or more securing apertures 130 for receiving one or more securing members.

A growth-enhancing layer 118 may be positioned on a least a portion of a surface of either or both of the first and second sealing members 114, 116. The growth-enhancing layer 118 may comprise collagen and/or one or more other tissue-like materials that provide a framework allowing the tissue to grow around and through the material. The growth-enhancing layer 118 may be positioned on each respective sealing member 114, 116 so that it may fill the fistula 106 when the healing device 110 is operably connected to the tissue 112.

FIGS. 3A-3C are cross-sectional, top plan and bottom plan views, respectively, of the healing device 110 operably connected to tissue 112 surrounding the large-opening fistula 106. As shown in FIG. 3A, the first and second sealing members 114, 116 may be positioned on opposite sides of the tissue 112, so that each side or opening (e.g., proximal and distal openings) of the large-opening fistula 106 may be substantially covered. Additionally, each sealing member 114, 116 may be configured to extend over the large-opening fistula 106 to cover the margins of the tissue 112 surrounding the large-opening fistula 106.

As can be seen best in FIG. 3A, the growth-enhancing layer 118 is positioned on the sealing members 114, 116 so that it may be inserted into the large-opening fistula 106. For example, the growth-enhancing layer 118 may be positioned on a distal surface of the first sealing member 114 and the first sealing member 114 may be operably connected to the tissue 112 so that its distal surface faces the tissue 112. Similarly, the growth-enhancing layer 118 may be positioned on a proximal surface of the second sealing member 116 and the second sealing member 116 may be operably connected to the tissue 112 so that its proximal surface faces the tissue 112. This may allow the growth-enhancing layer 118 to partially fill the opening 106, thereby acting as a plug or cork for the opening 106.

To secure the two sealing members 114, 116 together, a tensioning cord 124 may be operably connected to the first sealing member 114 and fixed to the second sealing member 116 (see, e.g., FIG. 3B). The tensioning cord 124 provides a mechanism to pull the second, interior (distal) sealing member 116 toward the first, exterior (proximal) sealing member 114, sandwiching the tissue 112 therebetween. The tensioning cord 124 may be used to tension the sealing members 114, 116 in place, thereby allowing one or more securing members 132 (FIG. 3A) to be used to operably connect the sealing members 114, 116 across the tissue 112. Alternatively, the tensioning cord 124 may be used to operably connect the two sealing members 114, 116 to each other, without the need for sutures. As shown in FIG. 3A, an end of the tensioning cord 124 may include a loop 126 or other member that may be used to grasp and pull the tensioning cord 124.

Referring again to FIG. 3A, to operably connect the healing device 110 to the tissue 112, the second sealing member 116 may be inserted through the large-opening fistula 106 (e.g., be being rotated, deformed, compressed, etc.). The second sealing member 116 may then be positioned to cover the large-opening fistula 106 on a first end and to extend under the margins of the surrounding tissue 112. Additionally, the second sealing member 116 may be positioned so that the growth-enhancing layer 118 is substantially aligned with the large-opening fistula 106.

Once the second sealing member 116 is positioned over the distal opening of the large-opening fistula 106, the tensioning cord 124 (which may already be operably connected to the second sealing member 116) may be inserted or pulled through the large-opening fistula 106. The tensioning cord 124 may be threaded through the cord aperture 128 in the first sealing member 114. The first sealing member 114 may then be positioned over the proximal opening of the large-opening fistula 106 and may extend over the margins of the surrounding tissue 112. As with the second sealing member 116, the growth-enhancing layer 118 may be positioned within the large-opening fistula 106. In other words, the first sealing member 114 may be positioned over the large-opening fistula 106 in a way that substantially aligns the growth-enhancing layer 118 within the large-opening fistula 106. A surgeon may then pull the tensioning cord 124 to pull the interior or distal sealing member 116 flush and tight against the inner wall 122 of the tissue 112.

Once the first and second sealing members 114, 116 are positioned over the large-opening fistula 106, the surgeon may provide an upward force on the tensioning cord 124, pulling it against the tissue between the first and second sealing members 114, 116. The surgeon may provide simultaneous downward pressure on the first sealing member 114 as well (e.g., by using his fingers). As the second sealing member 116 is displaced upwards, the tissue 112 may be compressed between the two sealing members 114, 116. Securing members 132 (e.g., sutures) may be inserted through the first sealing member 114, the tissue 112, and the second sealing member 116 to operably connect all three elements together in the tensioned or compressed position. The securing members 132 may be inserted through the securing apertures 130 on each the first sealing member 114. Alternatively, the surgeon may simply suture the first sealing member 114 to the second sealing member 116 about their perimeters. In this way, the sutures will pass through the margins of the tissue 112 and thereby hold the first and second sealing numbers 114, 116 in place. In some embodiments, at least some (e.g., all) securing members 132 that are used to hold the first and second sealing members 114, 116 in place may not pass through the tissue 112 at all when positioned at the target site. Rather, they may pass through the fistula 106. This may, for example, limit the likelihood of inadvertently forming new fistulas or causing other tissue damage by forming new holes in the tissue 112.

The securing members 132 (e.g., sutures and/or stitches) hold the first and second sealing members 114, 116 in place. The securing members 132 may be positioned so that the tensioning force exerted by each securing member 132 is exerted on the sealing members 114, 116, rather than the tissue 112. This may help prevent the securing members 132 from further damaging the tissue 112. Additionally, the use of multiple securing members 132 may distribute the tension force over a relatively large area, rather than the single, pinpoint location of just the tensioning cord 124. Further, the securing members 132 may be spaced apart from the fistula 106, where the tissue 112 may be torn or otherwise damaged. The healing device 110 thus offers a compelling alternative to other wound healing devices or methods that require suturing the wound closed. Such suturing may place significant tension and sheer forces on the margins of the tissue 112 around a wound, which may lead to further tearing of the tissue 112 (especially delicate tissues) and may further prevent or hinder tissue re-growth.

As the securing member 132 operably connects the first and second sealing members 114, 116 together in a tensioned configuration, the healing device 110 seals the large-opening fistula 106. This can be important in the context of enteroatmospheric fistulas, as negative pressure may help to promote healing of the abdominal wound 102. Without sealing the enteric fistula 106, negative pressure on the abdominal wound 102 would draw fluid and material out of the intestine or bowel through the fistula, thus contaminating the abdomen, which can lead to sepsis or other infection. Additionally, positioning the growth-enhancing layers 118 within the large-opening fistula 106 causes the growth-enhancing layers 118 to partially or fully plug the large-opening fistula 106 and to potentially promote growth of the tissue 112 within and across the large-opening fistula 106. For example, the growth-enhancing layers 118 may provide a structure for supporting and encouraging tissue growth within the large-opening fistula 106.

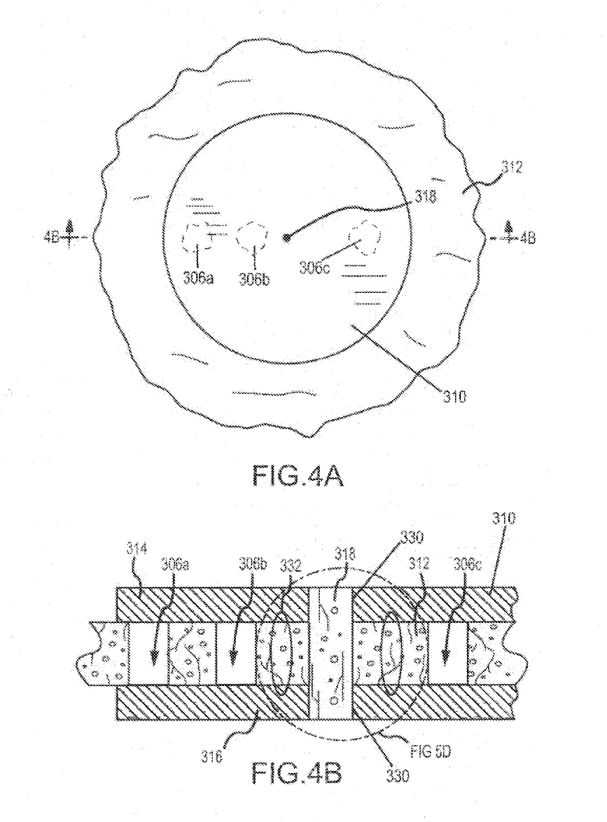

In another example, the healing device may be used to provide a tissue growth framework and protective layer for a tissue area with a large opening or multiple openings. For example, FIGS. 4A and 4B are a top plan view and a cross-sectional view, respectively, of a healing device 310 operably connected to tissue 312 (e.g., enteric tissue) having multiple fistulas 306a-306c and FIG. 4B is a cross-section view of the healing device 310 operably connected to the tissue 312 and substantially covering the fistula 306a-306c. The healing device 310 may include a first or exterior (proximal) sealing member 314 providing a first support surface and a second or interior (distal) sealing member 316 providing a second support surface. Each sealing member 314, 316 may include a plug aperture 330 for receiving a plug 318. The sealing members 314, 316 may extend over a portion of tissue 312 that may have multiple openings 306 dispersed throughout (e.g., due to wounds from shrapnel, birdshot, shattered glass, etc.). Thus, the healing device 310 may provide a tissue growth framework for multiple fistulas 306a-306c, while also protecting the multiple fistulas 306a-306c.

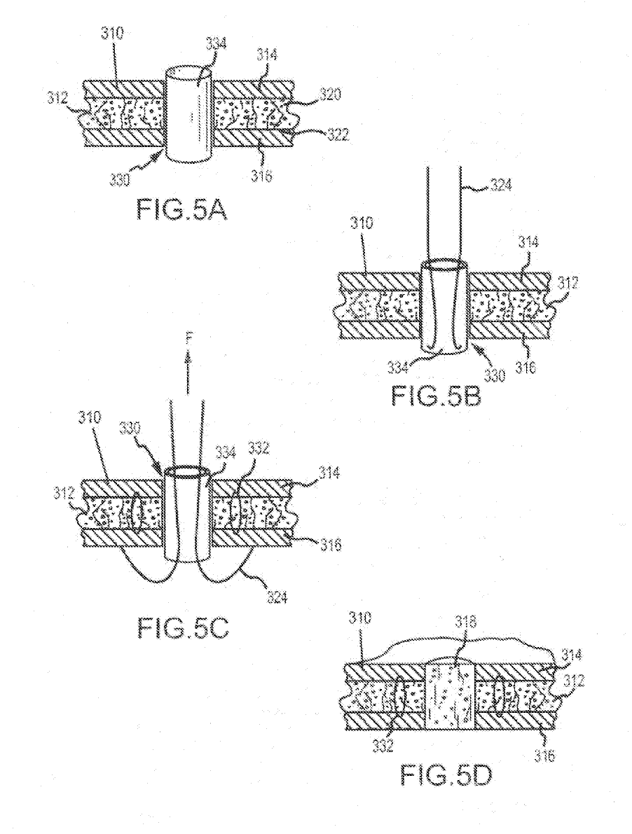

The healing device 310 may be positioned over the tissue 312 and one or more tensioning wires 324 (FIGS. 5B and 5C) may be used to support the interior or distal sealing member 316 while one or more securing members 332 (FIGS. 5C and 5D) may be used to secure the sealing members 314, 316 to each other and to the tissue.

FIG. 5A is an enlarged cross-sectional view of the healing device 310 positioned on the tissue 312 before the sealing members 314, 316 have been operably connected together. To operably connect the healing device 310 to the tissue, the second sealing member 316 may be positioned on a second surface 322 of the tissue 312 (e.g., by being deformed or compressed). Then, the first sealing member 314 may be positioned on a first surface 320 of the tissue 312 opposing the second sealing member 316. The two sealing members 314, 316 may be positioned on the tissue 312, such that the plug apertures 330 within each sealing member 314, 316 may be substantially aligned with the other.

Once the sealing members 314, 316 are aligned, an insertion shaft 334 may be inserted through the plug aperture 330 in each sealing member 314, 316 and through an opening in the tissue 312, such as a fistula wound opening or an opening created for the purpose of operably connecting the healing device 310 (e.g., via a needle, punch, or scalpel). The insertion shaft 334 may be a generally hollow cylindrical shaft that is configured to approximately the same diameter as the plug aperture 330. Other embodiments of insertion shafts having different sizes and/or configurations may also be used, as appropriate.

After the insertion shaft 334 has been inserted, the tensioning wires 324 may be inserted therein. FIG. 5B is a cross-sectional view of the healing device 310 positioned on the tissue 312 with the tensioning wires 324 partially inserted within the insertion shaft 334. The tensioning wires 324 may be resilient so that they may be bent to an insertion shape and then revert to an original shape. In one implementation, the tensioning wires 324 may be made of a shape memory metal (e.g., NiTiNol). For example, as shown in FIG. 5B, the tensioning wires 324 may be substantially straight as they are inserted through the insertion shaft 324. However, as shown in FIG. 5C, the tensioning wires 324 may later revert to a curved or hooked configuration that bends upwards towards a bottom surface of the second sealing member 316. FIG. 5C illustrates the tensioning wires 324 completely inserted through the insertion shaft 334 and abutting the second sealing member 316.

Once the tensioning members 324 are in place adjacent the interior sealing member 316, a tensioning force F may be provided to the tensioning members 324. For example, a surgeon may pull the tensioning members 324 proximally, creating the tensioning force F. The tensioning force F may pull each tensioning member 324 upward toward the first sealing member 314 and as, the tensioning members 324 are curved upward, they may pull the second sealing member 316 toward the first sealing member 314. Multiple securing members 332 may then be used to secure the first sealing member 314, through the tissue 312, to the second sealing member 316. For example, the surgeon may suture the two sealing members 314, 316 together. The tensioning members 324 allow the sealing members 314, 316 to be secured in a tensioned position against the tissue 312 so that the healing device 310 may seal the fistulas 306a-306c.

The securing members 332 may be sutures, staples, or other connection devices that are passed through the sealing members 314, 316 and the tissue 312. While not shown here, in some cases a securing member 332 may couple a sealing member to tissue without also coupling the sealing member to another sealing member. In some embodiments, the securing members 332 may be positioned away from the margins of the damaged tissue 312 (i.e., away from the fistulas 306a-306c). The sealing members 314, 316 cover and seal the fistulas 306a-306c and may be held in place using only minimal sutures or other securing members 332, thus reducing the additional punctures to the tissue. As discussed above with respect to the healing device 110 illustrated in FIG. 3A, the securing members 332 may provide tension between the sealing members 314, 316, without also placing force on the damaged tissue 312, thereby mitigating further damage.

FIG. 5D is a cross-sectional view of the healing device 310 operably connected to the tissue 312 with a plug 318 inserted in the place of the insertion shaft 334. More specifically, after the sealing members 314, 316 have been secured in place via the securing members 332, the insertion shaft 334 may be removed. Once the insertion shaft 334 is removed, an aperture 330 through the sealing members 314, 316 and the tissue 312 may remain. The plug 318 may then be inserted through the plug aperture 330 within both sealing members 314, 316, as well as through the tissue 312. The plug 318 may comprise collagen and/or one or more other tissue growth-enhancing materials. Thus, the plug 318 may provide a framework for the tissue 312 to grow through and around, thereby filling the aperture 330 left by the insertion shaft 334. In alternative embodiments, the plug 318 may not go completely through the sealing members 314, 316. In other alternative embodiments, the plug 318 may also be a non-biologic plug used to secure the sealing members 314, 316. In still other embodiments, the plug 318 may be a solid rod or sliding tapered rod or other eleasable locking designed to lock and release one or both disks. The aperture 330 filled by the plug 318 may be configured to be substantially smaller that the openings 306a-306c and thus may not substantially damage the tissue 312. While a plug 318 is used here, in some cases, a bioabsorbable insertion shaft 334 and/or an insertion shaft that promotes tissue in-growth may alternatively be used, or may be used in conjunction with a plug.

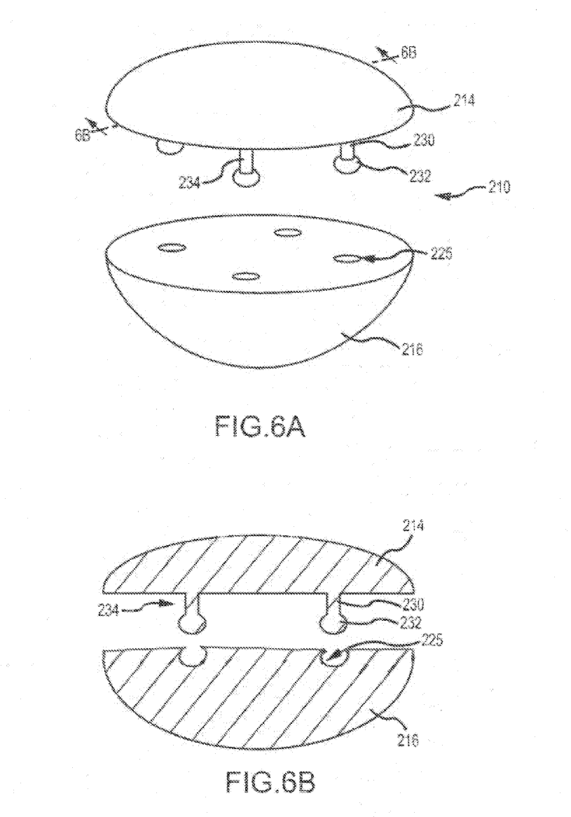

FIGS. 6A and 6B are exploded isometric and cross-sectional views, respectively, of another embodiment of the healing device 210. The healing device 210 may include a first sealing member 214 and a second sealing member 216 configured to be operably connected together. Additionally, a set-off between the first sealing member 214 and the second sealing member 216 may be provided to create a gap or space between the two sealing members 214, 216. The two sealing members 214, 216 act to secure a growth-enhancing material within a large fistula 106 in the tissue 112, as well as to provide a covering for the fistula 106. Additionally or alternatively, the two sealing members 214, 216 may protect the fistula and thereby promote healing growth of the tissue. The two sealing members 214, 216 are configured to operably connect to tissue 112 surrounding the fistula 106.

The first sealing member 214 may be a disk or semi-circular shape and is configured to be positioned over the fistula 106 so as to substantially cover the fistula 106. The first sealing member 214 may also have a diameter larger than a diameter of the fistula 106, so that it extends over the tissue margins surrounding the fistula 106. Additionally, the first sealing member 214 may comprise one or more bioabsorbable materials and may have an impermeable coating, so that fluids, bacteria, or other materials may be substantially prevented from ingress and egress into and out of the fistula 106 when the healing device 210 is operably connected to the tissue 112.

Fastening or securing members 234 may extend from a bottom surface of the first sealing member 214. The fastening members 234 may include a stem 230 and a head 232 formed at a bottom end of the stem 230. The stem 230 may be a substantially cylindrical member and the head 232 may be a ball or semi-spherical shape. Other appropriate stem or head shapes and configurations may also be used. For example, a sliding tapered rod or other releasable locking member may be used (not illustrated), such that, when removed, it allows 232 to shrink in diameter. When moved forward, the sliding tapered rod like member may expand 232 to lock it into the receiving cavities 225.

The stem 230 may have a length approximately the same as a thickness of the tissue 112 so that the stem 230 extends through the tissue 112 and operably connects to the second sealing member 216. The head 232 is configured to be received within a corresponding cavity in the second sealing member 216. The head 232 may function as a keyed structure that fits within a corresponding shape of the opposing cavity within the second sealing member 216. While not shown here, in some cases, the second sealing member 216 may alternatively or additionally comprise one or more structures that fit within one or more corresponding cavities in the first sealing member 214.

The second sealing member 216 may have a disk or semi-circular shape that may be similar to or substantially the same as the first sealing member 214. Receiving cavities 225 are defined within an upper surface of the second sealing member 216. The receiving cavities 225 are configured to receive the heads 232 of the fastening members 234 in a snap-fit arrangement. For example, the receiving cavities 225 may be generally semi-spherically shaped to cradle and contain the ball-shaped heads 232 of the fastening members 234. Other suitable shapes may also be used.

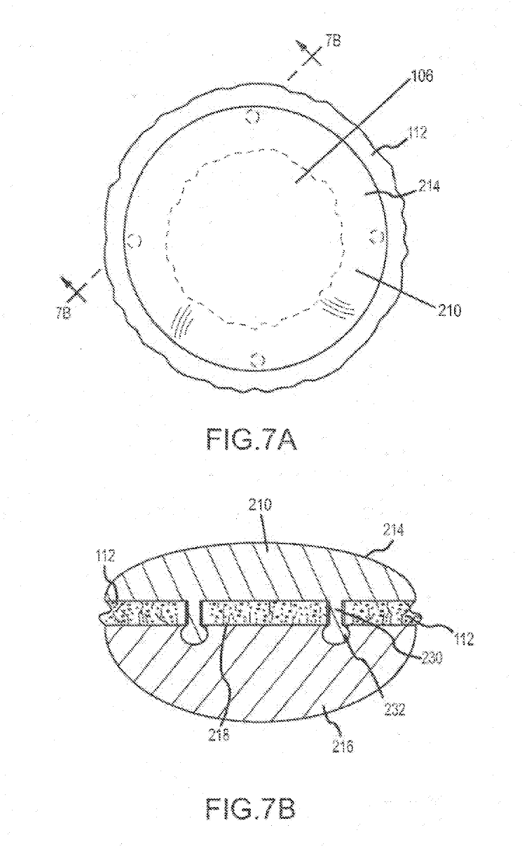

FIGS. 7A and 7B are top plan and cross-sectional views, respectively, of the healing device 210 operably connected to the tissue 112 and covering the fistula 106. The first and second sealing members 214, 216 may be operably connected together and may secure a growth-enhancing layer 218 within the fistula 106.

The second sealing member 216 may be rotated, angled, compressed, or otherwise deformed and then may be inserted into the fistula 106 to be placed on the interior (distal) side of the tissue 212 (e.g., within an enteric cavity). The second sealing member 216 may then be positioned such that the receiving cavities 225 may be substantially aligned with and face the interior opening of the fistula 106. Additionally, at least a portion of the second sealing member 216 may be configured to extend over the tissue 112 surrounding the fistula 106. Once the second sealing member 216 is positioned adjacent the tissue 112, the growth-enhancing layer 218 may be positioned within the fistula 106 between the receiving cavities 225 on the second sealing member 216. The first and second sealing members 214, 216 may be press-fit together by applying pressure to each of their outer surfaces, or may be coupled to each other using any other suitable method. For example, in some embodiments, the second sealing member 216 may have a pulling member coupled thereto, and the pulling member may be pulled upon (e.g., while the first sealing member 216 is pushed upon) to bring the first and second sealing members together. The first sealing member 214 may thus be operably connected to the second sealing member 216 by inserting the head 232 of each stem 230 into a corresponding receiving cavity 225. The heads 232 and the receiving cavities 225 may operably couple together via a snap-fit or other appropriate fastening mechanism. In the embodiments described above, the snap-fit connections are also releasable, so that the sealing members 214, 216 may be released from one another.

As can be seen in FIGS. 7A and 7B, the healing device 210 positions the growth-enhancing layer 218 within the fistula 106 and secures the growth-enhancing layer 218 in place. In some alternatives, the growth-enhancing layer 218 may be coupled to or integral with one or both of the first and second sealing members 214, 216. In certain embodiments, one or more fastening members 234 may be inserted into the fistula opening 106 in the tissue. Alternatively or additionally, one or more fastening members 234 may be inserted into tissue surrounding the fistula opening 106. In some embodiments, one or more fastening members 234 may be inserted through the opening of the fistula 106 and used to help contain the seal within the opening, without passing through tissue (and thereby creating new holes in the tissue).

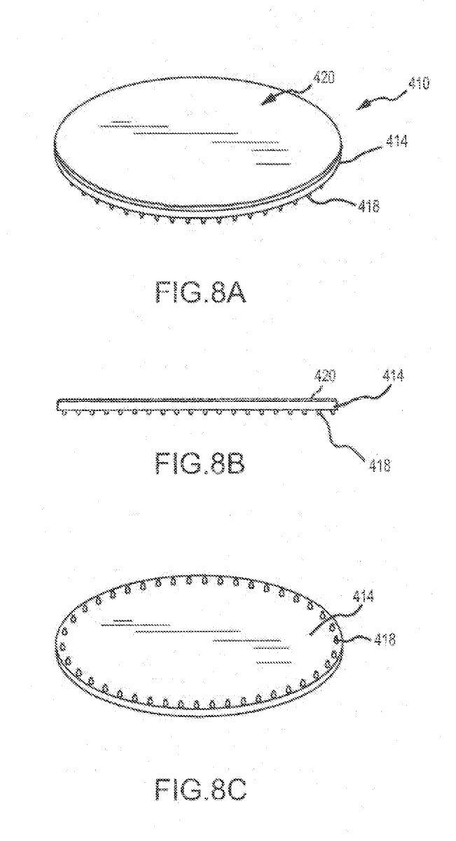

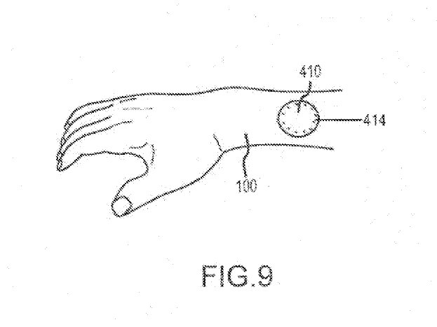

FIGS. 8A and 8B are isometric and side elevational views, respectively, of a fourth embodiment of a healing device 410. FIG. 8C is a bottom isometric view of the healing device 410. The healing device 410 may provide a cover and protection member for a tissue opening, while also providing a tissue growth framework by spanning a wound within the skin of the person 100. As shown in FIGS. 8A-8C, the healing device 410 may include a sealing member 414 having a plurality of support feet 418 spaced apart around a perimeter. The healing device 410 is configured so that at a first stage it may be operably connected to the tissue 112 by an attachment member (e.g., tape, gauze, sutures, etc.). Then, the healing device 410 is configured so that the support feet 418 may ingress within the tissue 112 and the tissue 112 may bond with or partially envelop the healing device 410. FIG. 9 shows the healing device 410 in place on an arm of a patient.

The sealing member 414 may be relatively thin and may have any suitable shape. In some embodiments, the sealing member 414 may be formed of one or more bioabsorbable materials. In certain embodiments, an outer surface of the sealing member 414 may be coated with an impermeable coating layer 420. The sealing member 414 is configured to be positioned over, and to substantially cover, a tissue or wound opening 102. For example, as shown in FIG. 9, the sealing member 414 may have a diameter larger than the wound opening 102, so that the wound opening 102 may be substantially covered.

The support feet 418 may comprise one or more bioabsorbable materials, and are configured to support the sealing member 414 on the tissue 112. The support feet 418 provide multiple pressure points on the tissue 112. As the healing device 410 is continuously held against the tissue 112 under pressure (via the attachment member), the pressure points exerted by the feet 418 may cause the tissue 112 to re-configure and envelope a portion, if not all, of the feet 418. This may cause the feet 418 to “sink” into the tissue. For example, when the healing device 410 is first coupled to the tissue 112, the sealing member 414 may be positioned substantially over the wound opening 102, with the perimeter of the healing device 410 extending over the margins of a first surface 120 of the tissue 112. Then, after the feet 418 have been substantially received within the tissue 112, the sealing member 414 may be substantially aligned with or rest on a portion of the first surface 120 of the tissue 112. The bioabsorbable material of the feet 418 may promote tissue bonding at small points in the tissue 112 surrounding the wound 102 and may act as sutures or other securing members. Further, the bioabsorbable material of the sealing member 414 is provided to promote new tissue growth across the wound opening 102. In some implementations, additional collagen or other tissue growth frameworks may be placed in the wound 102 under the healing device 410, in order to further promote new tissue growth over the wound 102. Additionally, the impermeable coating layer 420 may prevent the healing device from degrading or decomposing too quickly due to exposure to outside agents, such as soap and water, dust, dirt, chemicals, etc.

The foregoing description has broad application. For example, while embodiments disclosed herein may focus on closing larger fistulas or multiple fistulas in a condensed area, the concepts disclosed herein may equally apply to closing other type of wounds and openings. Similarly, although the tissue openings and applications may be discussed with respect to humans, the devices and techniques disclosed herein are equally applicable to other animals. Accordingly, the discussion of any embodiment is meant only to be exemplary and is not intended to suggest that the scope of the disclosure, including the claims, is limited to these examples.

All directional references (e.g., proximal, distal, upper, lower, upward, downward, left, right, lateral, longitudinal, front, back, top, bottom, above, below, vertical, horizontal, radial, axial, clockwise, and counterclockwise) are only used for identification purposes to aid the reader's understanding of the present disclosure, and do not create limitations, particularly as to the position, orientation, or use of this disclosure. Connection references (e.g., attached, coupled, connected, and joined) are to be construed broadly and may include intermediate members between a collection of elements and relative movement between elements unless otherwise indicated. As such, connection references do not necessarily infer that two elements are directly connected and in fixed relation to each other. The exemplary drawings are for purposes of illustration only and the dimensions, positions, order and relative sizes reflected in the drawings attached hereto may vary.

The above specification, examples and data provide a complete description of the structure and use of exemplary embodiments of the invention as defined in the claims. Although various embodiments of the claimed invention have been described above with a certain degree of particularity, or with reference to one or more individual embodiments, those skilled in the art could make numerous alterations to the disclosed embodiments without departing from the spirit or scope of the claimed invention. For example, features disclosed with respect to one embodiment may be used in other embodiments, as appropriate. Other embodiments are therefore contemplated. For example, in some embodiments, an integral, one-piece or unitary device may be used to treat tissue openings or wounds. The device may be placed in a tissue opening in an elongated configuration, and may then be converted into a configuration in which it is no longer elongated and instead forms a double-lipped seal. In certain embodiments, a device described herein may comprise one or more shape-memory and/or super-elastic materials, such as Nitinol. This may allow the device to be inserted into a target site in one configuration (e.g., straight), and to later convert into a shape in which the device comprises two sealing members that are held together. It is intended that all matter contained in the above description and shown in the accompanying drawings shall be interpreted as illustrative only of particular embodiments and not limiting. Changes in detail or structure may be made without departing from the basic elements of the invention as defined in the following claims.

What is claimed is:

1. A healing device configured for sealing at least one fistula in tissue, the healing device comprising:
a first sealing member configured for placement over a first opening of the at least one fistula in a first surface of the tissue;
a second sealing member configured for placement over a second opening of the at least one fistula in a second, opposing surface of the tissue; and
a first growth-enhancing layer coupled to a portion of at least one of the first sealing member or the second sealing member.

2. The healing device of claim 1, further comprising one or more securing members connected to the first sealing member and the second sealing member and configured to secure the at least one fistula between the first and second sealing members.

3. The healing device of claim 2, wherein the one or more securing members are further configured to secure margins of the tissue surrounding the at least one fistula between the first and second sealing members.

4. The healing device of claim 1, further comprising a tensioning cord fixedly connected to the second sealing member.

5. The healing device of claim 4, wherein:
the first sealing member defines an aperture for passage of the tensioning cord through one of the at least one fistula or the aperture; and
the tensioning cord is configured for applying a tensioning force on the second sealing member.

6. The healing device of claim 1, wherein the first growth-enhancing layer comprises at least one polymer selected from the group consisting of collagen, polyglycolic acid, polylactic acid, poly-L-lactide acid, polyglycolic-lactic acid, poly-DL lactic acid, and combinations thereof.

7. The healing device of claim 1, wherein the first growth-enhancing layer comprises collagen.

8. The healing device of claim 1, wherein:
the first growth-enhancing layer is coupled to a surface of the first sealing member; and
the healing device further comprises a second growth-enhancing layer coupled to a surface of the second sealing member.

9. The healing device of claim 1, wherein:
the first sealing member comprises a first plug aperture; and
the second sealing member comprises a second plug aperture.

10. The healing device of claim 9, wherein the plug is further configured for insertion into the first plug aperture and the second plug aperture with passage through one of the at least one fistula.

11. The healing device of claim 1, further comprising:
at least one fastening device coupled to a surface of the first sealing member; and
at least one receiving structure in the second sealing member,
wherein the at least one receiving structure is configured to couple with the at least one fastening device.

12. The healing device of claim 11, wherein the at least one fastening device comprises:
a stem extending from the surface of the first sealing member; and
a head extending from a distal end of the stem.

13. A wound closure device for closing a tissue wound, the wound closure device comprising:
a first sealing member configured for placement over a first region of a tissue wound and configured to extend beyond the tissue wound to cover margins of the tissue surrounding the tissue wound;
a second sealing member configured for placement over a second region of the tissue wound and configured to extend beyond the tissue wound to cover margins of the tissue surrounding the tissue wound; and
a first layer coupled to at least one of the first sealing member and the second sealing member,
wherein the first layer comprises allograft or at least one polymer selected from the group consisting of collagen, polyglycolic acid, polylactic acid, poly-L-lactide acid, polyglycolic-lactic acid, poly-DL lactic acid, or a combination thereof.

14. The wound closure device of claim 13, further comprising a coupling member coupled to the second sealing member and configured to apply a force to the second sealing member to move the second sealing member toward the first sealing member.

15. The wound closure device of claim 14, wherein the coupling member comprises a tensioning member configured to apply a tensioning force to the second sealing member to move the second sealing member toward the first sealing member.

16. The wound closure device of claim 13, further comprising a second layer, wherein the first layer is coupled to the first sealing member, and the second layer is coupled to the second sealing member.

17. The wound closure device of claim 16, wherein the first layer comprises collagen.

18. The wound closure device of claim 16, wherein the second layer comprises allograft or at least one polymer selected from the group consisting of collagen, polyglycolic acid, polylactic acid, poly-L-lactide acid, polyglycolic-lactic acid, poly-DL lactic acid, or a combination thereof.

19. The wound closure device of claim 18, wherein the second layer comprises collagen.

20. The wound closure device of claim 13, wherein the first layer is a plug configured to be inserted in the tissue wound.

21. A method of closing a wound, the method comprising:
positioning a section of tissue defining a wound between a first sealing member and a second sealing member;
providing tension between the first sealing member and the second sealing member across the intermediate tissue; and
coupling the first sealing member and the second sealing member together across the tissue to seal the wound and contact the intermediate tissue.

22. The method of claim 21, wherein:
a plurality of fastening members extend from a surface of the first sealing member;
the second sealing member comprises a plurality of respective receiving structures configured to receive corresponding fastening members; and
the method further comprises engaging each of the plurality of fastening members with respective ones of the plurality of receiving structures.

23. The method of claim 21, wherein:
a tensioning cord is coupled to the second sealing member; and
providing tension between the first sealing member and the second sealing member further comprises providing a force on the tensioning cord to pull the second sealing member toward the first sealing member.

24. The method of claim 23, wherein the method comprises inserting a securing member to couple the first sealing member and the second sealing member.

25. The method of claim 23, wherein:
the first sealing member comprises a first plug aperture;
the second sealing member comprises a second plug aperture; and
providing tension between the first sealing member and the second sealing member further comprises:
inserting an insertion shaft into the first plug aperture, through the wound, and through the second plug aperture;
inserting a tensioning member through the insertion shaft;
positioning a distal end of the tensioning member against a surface of the second sealing member; and
providing a tensioning force on the tensioning member to pull the second sealing member toward the first sealing member.

26. The method of claim 25, wherein the tensioning member comprises a tensioning wire.

27. The method of claim 25, wherein the method comprises inserting a securing member to couple the first sealing member and the second sealing member.

28. A wound sealing device comprising:
a first sealing member; and
a plurality of feet extending from a bottom side of the first sealing member and comprising one or more bioabsorbable materials,
wherein the feet are configured to be enveloped in and bond with tissue surrounding a wound after the wound sealing device is held over the wound and against the tissue under pressure for an extended period of time.

29. The wound sealing device of claim 28, wherein the first sealing member comprises one or more bioabsorbable materials.

30. The wound sealing device of claim 28, wherein the first sealing member comprises one or more biocompatible materials.

31. The wound sealing device of claim 28, wherein the sealing device comprises one or more implant-grade materials.

32. The sealing device of claim 28, wherein the sealing device comprises one or more resorbable materials that are capable of being resorbed into the tissue over time.

33. The wound sealing device of claim 28, further comprising an impermeable layer coating a side of the first sealing member.

34. The wound sealing device of claim 33, wherein the side of the sealing member is configured to contact the tissue during use.

35. The wound sealing device of claim 28, further comprising a second sealing member.

36. The wound sealing device of claim 35, further comprising a tissue growth promoting material positioned on both of the first and second sealing members.

37. The wound sealing device of claim 35, further comprising a tissue growth promoting material positioned on the second sealing member.

38. The wound sealing device of claim 28, further comprising a tissue growth promoting material positioned on the first sealing member between the plurality of feet.

39. The wound sealing device of claim 28, wherein the plurality of feet are arranged about a perimeter of the first sealing member.
